(19) United States
(12) Patent Application Publication
(10) Pub. No.: US 2003/0086064 A1
(43) Pub. Date: May 8, 2003

(54) Head mounted infrared fundus illuminator

(76) Inventor: Erich E. Sutter, Redwood City, CA (US)

Correspondence Address: Erich E. Sutter, 711 PALOMAR DR., REDWOOD City, CA, 94062, US

(21) Appl. No.: 10/287,756

(22) Filed: Nov. 3, 2002

Related U.S. Application Data
(60) Provisional application No. 60/338,255, filed on Nov. 6, 2001.

(51) Int. Cl. A61B 3/10

(52) U.S. Cl. 351/221

(57) ABSTRACT

A small, lightweight infrared light emitting retinal illuminator is used to illuminate the fundus of the eye without illuminating the cornea. It is mounted to the head near the eye with adhesive tape or a double sided, adhesive patch (FIG. 4). The IR radiation of the device is directed toward the skin surrounding the eye, preferably just outside the temporal juncture of the upper and lower eyelids. It penetrates the skin and the sclera of the eye and provides sufficient IR radiation to the fundus of the eye for obtaining images of the retina with IR-sensitive cameras.

### GOVERNMENT CONTRACT

The U.S. government has rights in the claimed invention pursuant to NIH Grant EY-6861.

### CROSS-REFERENCE TO RELATED APPLICATION

This application is entitled to the benefit of Provisional Patent Application Ser. No. 60/338,255 filed Nov. 6, 2001.

### BACKGROUND OF THE INVENTION

1. Field of Invention

This invention relates to real-time imaging and monitoring of the fundus of human and animal eyes using infrared radiation. Specifically, it provides a simplified and economical method of illuminating the fundus.

2. Description of Prior Art

The fundus of the eye is the surface in the back of the eye on which the lens projects an image of the outside world. It is covered with the retina which consists of neural tissue with a layer of light sensitive cells. In science and medicine it is often necessary or desirable to monitor the retina of a human or animal eye using infrared light while the eye is looking at a stimulus. For example, in an ophthalmology clinic or in biomedical research, one may need to monitor the location of a stimulus on the retina of the patient's eye. An important application of fundus imaging monitors how well a patient fixates on a prescribed target while a multifocal electroretinogram [mfERG] is derived from the eye (ref). In order not to interfere with the stimulation, the image has to be obtained using radiation in the invisible portion of the spectrum, usually in the near infrared (IR). An image of the fundus can be obtained through the pupil of the eye by focusing an appropriately designed camera on the retina. For this purpose, the retina has to be adequately illuminated. In the past the necessary light or IR radiation has been provided through the pupil. In this approach, the pupil opening is shared by the light or radiation entering as well as exiting. Since reflections from the cornea can severely degrade the image, the optics in conventional fundus cameras separate the entrance and exit paths of light. Usually, the illuminating radiation is provided through an annulus covering the periphery of the pupil. Only the return light exiting through the central area of the pupil is used for imaging. This optical design is fairly complex and expensive.

Another solution to fundus imaging uses a scanning laser ophthalmoscope (SLO). This instrument scans the retina with a laser beam through the pupil and assembles the fundus image from the reflected light. In this case the image degrading corneal reflections are greatly reduced. Each point on the scan path is only affected by the corneal reflection from the light illuminating it. SLOs are relatively complex and very expensive.

When imaging in the near IR spectral range is desired, another approach is feasible. IR relatively easily penetrates through human and animal tissue. Thus illuminating radiation can be supplied through the sclera of the eye rather than the pupil. If none of the radiation is passed through the pupil, image degrading corneal reflections are avoided. In addition, all the return light through the entire pupil area can be used for imaging. This method cannot be used when retinal illumination in the visible region of the spectrum is required, since such light does not sufficiently penetrate through the tissues.

Pomerantzeff in U.S. Pat. No. 4,200,362 shows an apparatus for irradiating the sclera of the eye with IR radiation for illuminating the fundus of the eye. Such radiation reaches the fundus with sufficient intensity to obtain an image of the retina. Pomerantzeff's and other methods for IR illumination of the fundus suffer from the following disadvantages:

1. The source of the radiation is separate from the subject's head. Therefore, the retinal illumination is affected by head movements.

2. The illuminating radiation is focused on the sclera. Thermal heating caused by the IR radiation dries the tear film causing discomfort and/or blinking.

3. The devices of the prior art are complex and expensive to manufacture.

4. The devices of the prior art require elaborate cleaning and maintenance procedures.

5. As IR radiation is applied remotely, a fraction of the radiation may fall on the cornea causing image degrading reflections.

6. The devices of the prior art cannot be used when the sclera is covered by the speculum of conventional contact lens electrodes used in ERG recording.

### OBJECTS AND ADVANTAGES

Accordingly, besides the object and advantages of the fundus illumination through the sclera described in the patent of Pomerantzeff, several objects and advantages of the present patent are:

1. to attach the source of the radiation to the subject's head, thus eliminating all changes in retinal illumination due to head movements.

2. to focus the illuminating radiation on the skin rather than the cornea. Thus, any thermal heating it causes does not dry the tear film causing discomfort and/or blinking.

3. to provide a device that can be manufactured very inexpensively.

4. to provide an IR emitting device that can easily be disinfected between uses or, because of its low cost, can be disposed after use.

5. to apply the IR radiation through IR emitters in contact with the skin. Thus no significant amount of radiation reaches the optics of the camera from areas other than the pupil. This greatly reduces image degradation due to dust and smudges on the imaging optics.

6. to make it possible to use IR illumination through the skin and the sclera when most of the sclera is covered by the speculum of conventional ERG electrodes. One commonly used electrode with such a speculum is the Burian-Allen electrode.

### SUMMARY

The fundus illuminator of the invention is a lightweight IR emitting device. It can be mounted to the head near the eye with adhesive tape or a double sided, adhesive patch. The IR radiation of the device is directed toward the skin surrounding the eye. The device provides sufficient IR radiation to the fundus of the eye for retinal imaging.

The invention of the present patent differs in three important ways. First, the IR radiation is supplied by means of small and inexpensive IR emitting diodes. Second, the device is mounted on the head of the subject near the eye. Third, the radiation is focused on the eyelid rather than directly on the sclera or the cornea. This results in several significant advantages.

### DESCRIPTION OF DRAWINGS

FIG. 1 shows an assembly of two IR emitting diodes and wire leads for illuminating the fundus according to the invention.

FIG. 2 shows how the diode wire leads of FIG. 1 are bent.

FIG. 3A shows the assembly of FIG. 1 after first dipping in a synthetic rubber coating with a plastic disc attached.

FIG. 3B shows the assembly of FIG. 1 with an attached plastic disc after a second coating with synthetic rubber.

FIG. 3C shows the coated assembly with openings cut for the IR diode lenses.

FIG. 4 is a schematic drawing showing a person's head with the device of the invention mounted.

FIG. 5 is a schematic drawing showing a cross-section through the right eye and temple of the person of FIG. 4 with the device of the invention mounted.

### REFERENCE NUMERALS IN DRAWINGS

1. IR emitting diodes

2. Wire leads of diodes with solder junctions

3. Wire leads

4. Synthetic rubber coating

5. Pliable plastic disc

6. Head mounted fundus illuminator

7. Eye

8. Temple

9. Bone

10. Bridge of nose

11. Current source

22 Skin of the eyelid

23 Sclera

24 Cornea

### DESCRIPTION

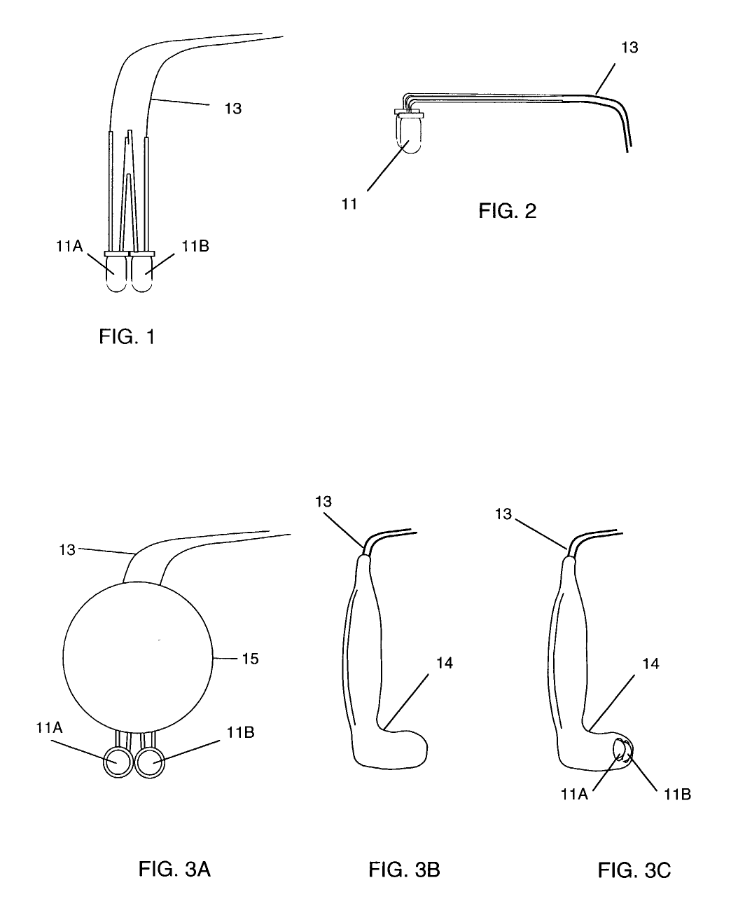

Preferred Embodiment—FIGS. 1, 2, 3A, 3B, and 3C

1. A fundus illuminator, in accordance with the invention, comprises two IR diodes. Two IR emitting diodes 11A and 11B (FIG. 1) are soldered in series so that an anode lead of diode 11A is connected to the cathode lead of diode 11B. Diodes 11 preferably are selected to emit light having a wavelength between 780 nm and 85 nm. Two flexible isolated wires 13 are soldered respectively to the two remaining diode leads, i.e., to the cathode of diode 11A and the anode of diode 11B (FIG. 1)

2. The leads 13 (FIG. 2) of the IR emitting diodes 11 are bent at right angles to the axis of the diodes.

3. The diodes and their leads are coated up to and including the solder connections with a flexible coating (not shown). This can be accomplished, e.g., by dipping into a solution of synthetic rubber, such as those sold under the trademarks PolyShield or Plastidip.

4. While the dipping rubber is still soft and sticky, the leads are pressed onto a thin, pliable plastic disc 15 about 32 mm in diameter (FIG. 3A).

5. The diodes with attached disc 15 are dipped again in the coating solution and suspended for drying (FIG. 3B) of the coating 14. The coating should be allowed to cover and form a relatively thick coating 14 over the diodes 11, their leads, disc 15 and part of wire 13 as shown.

6. While coating 14 is still soft, small openings are cut into the coating over diodes 11 to expose the tips of the diodes from which the IR radiation is emitted via internal lenses (not shown) (FIG. 3C).

The following description of the retinal IR illuminator and its construction contains many specificities. However, these should not be construed as limitations on the scope of the invention, but rather as an exemplification of one preferred embodiment and application thereof.

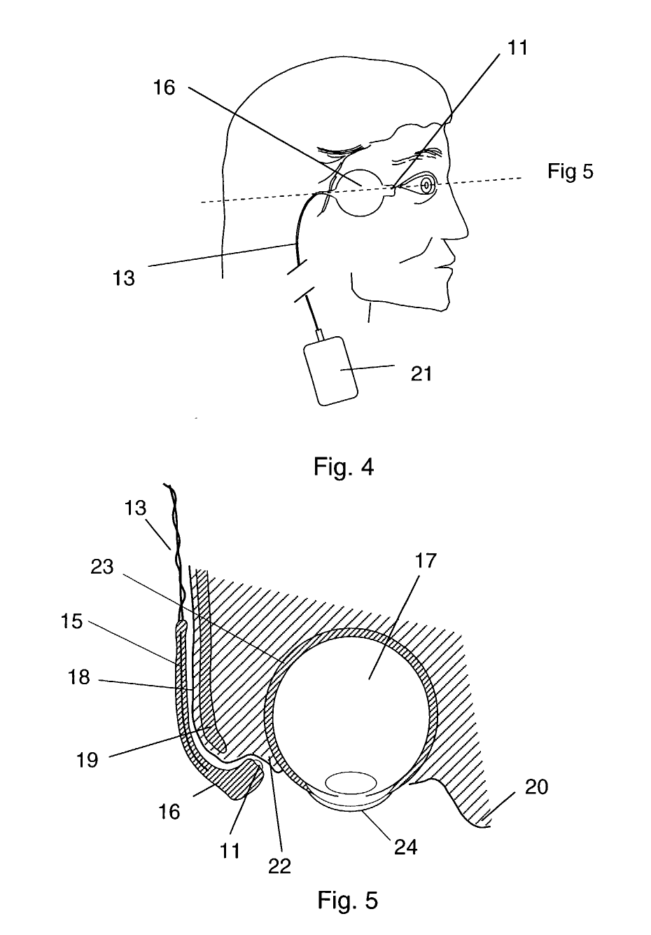

Operation of the Invention—FIGS. 4 and 5

The resulting device is electrically well insulated by the coating 14. The thin disk is soft, pliable, and rubbery to the touch. When applied to the head of a person (FIG. 4), it is easily shaped and bent to fit the curvature of the temple (FIG. 5). The lenses of diodes 11 are positioned so that they slightly touch the skin adjacent to the subject's eyelids 22 at the temporal corner of the eye. The leads of diodes 11 provide the necessary stiffness to disc 15 so that it maintains its shape when bent. Wire leads 13 of the device are connected to a suitable adjustable current source 21. The preferred location for irradiation is the temporal corner just outside the juncture of the upper and lower eyelids (FIG. 4). The device is attached to the head in this location by means of adhesive tape such as surgical tape used in clinics or a double sided adhesive pad.

Alternatively, the device can be attached to the cheek. Diodes 11 are positioned above the cheekbone and shine through the lower eyelid.

In some animals, other locations produce better results. For instance, in rodents such as rats and mice the IR fundus illuminator can be placed under the jaw of the animal.

As the device may come in contact with tear fluid of patients, it must be either discarded or disinfected after each use. As it is coated and hermetically sealed in synthetic rubber, it can be disinfected. This is accomplished by submerging it in 10% chlorine solution or another disinfecting agent. When mass-produced, the device can be manufactured inexpensively, making it disposable.

I claim:

1. A device for illuminating the fundus of the eye for the purpose of imaging said fundus comprising:
a source of radiation in the near infrared range of the spectrum positioned at a location on the skin surrounding the eye so that a portion of said radiation passes through said skin and the sclera of the eye,
said radiation having sufficient intensity to illuminate said fundus, whereby said fundus is illuminated so that no part of said radiation will fall on the cornea of said eye.

2. The device of claim 1 wherein said source of radiation comprises at least one infrared emitting diode.

8. A device for illuminating the fundus of the eye for the purpose of imaging said fundus comprising:
means for providing optical radiation in the near infrared range of the spectrum,
means for mounting said source so that infrared radiation from said source impinges on the skin adjacent and beyond the temporal corner of the eye, said source having sufficient intensity so that said infrared radiation from said source passes through said skin, the outer covering of said eye, and the internal filling of said eye and impinges on the retina of said eye without falling on the cornea of said eye, whereby said retina will be illuminated by said radiation without causing reflections from said cornea interfering with light exiting from the pupil of said eye.

9. The device of claim 8 wherein said means for providing said optical radiation comprises two infrared light-emitting diodes positioned adjacent each other.
